(19) United States
(12) Patent Application Publication
(10) Pub. No.: US 2014/0303424 A1
(43) Pub. Date: Oct. 9, 2014

(54) METHODS AND SYSTEMS FOR DIAGNOSIS AND TREATMENT OF NEURAL DISEASES AND DISORDERS

(71) Applicant: Iain GLASS, West Vancouver (CA)

(72) Inventor: Iain GLASS, West Vancouver (CA)

(21) Appl. No.: 14/216,361

(22) Filed: Mar. 17, 2014

Related U.S. Application Data
(60) Provisional application No. 61/793,488, filed on Mar. 15, 2013.

(51) Int. Cl. A61N 2/00

(52) U.S. Cl. CPC A61N 2/006; A61N 2/008
USPC 600/9

(57) ABSTRACT

The invention disclosed herein relates to methods and systems for modulating activity of a nervous system component. Neural pattern recognition is used to identify a neurological and/or psychiatric disease or disorder based on input generated by electric signals indicative of the subject's brain activity. In an embodiment, the method comprises receiving an input from one or more sensors, each sensor configured to sense a particular characteristic indicative of a neurological or psychiatric condition or state; developing treatment parameters based on the input received from the one or more sensors; and generating neural modulation signals for delivery to a nervous system component through one or more output devices in accordance with one or more developed treatment parameters.

### FIELD OF THE INVENTION

The invention disclosed herein generally relates to methods and systems for neural pattern recognition in a subject suffering from a neurological and/or psychiatric disease or disorder based on input generated by electric signals indicative of the subject's brain activity.

### BACKGROUND OF THE INVENTION

A number of techniques have been developed for controlling neurological conditions by electronically stimulating a pre-determined neurological region non-invasively. An example of such a technique is trancranial magnetic stimulation (TMS), which uses the principle of inductance to deliver electrical energy across the scalp and skull without the pain of direct percutaneous electrical stimulation.

In general, these procedures involve identifying a discrete region of the brain and focusing fluctuating magnetic fields generated by one or more coils positioned proximate the head at a location that induces electric current in the identified region of the brain. The type and character of the fluctuating magnetic field deposition, and the location of the targeted region of the brain typically depends on the type of therapeutic and/or diagnostic application that is to be achieved.

While such treatment modalities offer some benefits to patients, their efficacy is limited. Medical management using these techniques requires considerable iteration in dosing adjustments before an “optimal” balance between efficacy and side effect minimization is achieved. Variation, including both circadian and postprandial variations, causes wide fluctuation in symptomatology. With such treatment modalities it is difficult and often impossible to arrive at an optimal treatment “magnitude,” that is, an optimal dose or intensity of treatment. Furthermore, patients are subjected to periods of over-treatment and under-treatment due to variations in disease state.

Moreover, a particularly significant drawback to the above and other traditional treatment modalities is that they suffer from inconsistencies in treatment magnitude. For example, patient responsiveness to stimulation and augmentative pharmacologic agents, cause a change in response to a constant magnitude of therapy.

What is needed, therefore, is an apparatus and method for treatment of patients with neurological condition that is capable of determining and providing an optimal dose or intensity of treatment. Furthermore, the apparatus and method should be responsive to unpredictable changes in symptomatology and minimize alternations between states of over-treatment and under-treatment. The system should also be capable of anticipating future changes in symptomatology and neuromotor functionality, and being responsive to such changes when they occur.

### SUMMARY OF THE INVENTION

The invention described herein relates to a neurological control system for modulating activity of any component or structure comprising the entirety or portion of the nervous system, or any structure interfaced thereto, generally referred to herein as a “nervous system component.” The neurological control system generates neural modulation signals delivered to a nervous system component through one or more output devices in accordance with predetermined treatment parameters. Such treatment parameters can be based on input received by one or more sensors, each configured to sense a particular characteristic indicative of a neurological or psychiatric condition or state. In some embodiments, the neurological control system considers neural response, in the form of the sensory feedback, as an indication of neurological disease state and/or responsiveness to therapy, in the determination of treatment parameters.

Thus, some embodiments relate to a neural modulation system for use in treating disease which provides stimulus intensity that can be varied. The stimulation can be, for example, but not limited to, activating, inhibitory, and/or a combination of activating and inhibitory. The disease can be, for example, neurologic and psychiatric. For example, the neurologic disease can include, but is not limited to, Parkinson's disease, Huntington's disease, Parkinsonism, rigidity, hemiballism, choreoathetosis, dystonia, akinesia, bradykinesia, hyperkinesia, other movement disorder, epilepsy, or the seizure disorder. The psychiatric disease can include, for example, but is not limited to, depression, bipolar disorder, other affective disorder, anxiety, phobia, schizophrenia, multiple personality disorder. The psychiatric disorder can also include substance abuse, attention deficit hyperactivity disorder, impaired control of aggression, or impaired control of sexual behavior.

Some embodiments relate to a neurological control system. In some embodiments, the neurological control system modulates the activity of at least one nervous system component, and includes at least one non-invasive stimulating electrode, each constructed and arranged to deliver a neural modulation signal to at least one nervous system component; at least one sensor, each constructed and arranged to sense at least one parameter, including but not limited to physiologic values and neural signals, which is indicative of at least one of disease state, magnitude of symptoms, and response to therapy; and a control unit constructed and arranged to generate said neural modulation signal based upon a neural response sensed by said at least one sensor. In some embodiments, the neural modulation signal is based upon a neural response to a previously delivered neural modulation signal.

Some embodiments relate to an apparatus for modulating the activity of at least one nervous system component. The apparatus includes means for delivering neural modulation signal to a nervous system component; a means for sensing a particular characteristic indicative of a neurological or psychiatric condition or state. In one embodiment, the delivery means comprises a means for generating said neural modulation signal. The generating means can include a signal conditioning means for conditioning sensed neural response signals, wherein the conditioning can include, but is not limited to, amplification, lowpass filtering, highpass filtering, bandpass filtering, notch filtering, root-mean square calculation, envelope determination, and rectification; signal processing means for processing said conditioned sensed neural response signals to determine neural system states, including but not limited to a single or plurality of physiologic states and a single or plurality of disease states; and controller means for adjusting neural modulation signal in response to sensed neural response to signal.

Thus, in some embodiments, the neurological control system disclosed herein performs automated determination of the optimum magnitude of treatment. By sensing and quantifying the magnitude and frequency of brain activity in the patient, a quantitative representation of the level or “state” of the disease is determined. The disease state is monitored as treatment parameters are automatically varied, and the local or absolute minimum in disease state is achieved as the optimal set of stimulation parameters is converged upon. The disease state can be represented as a single value or a vector or matrix of values; in the latter two cases, a multi variable optimization algorithm can be employed with appropriate weighting factors. Automated optimization of treatment parameters expedites achievement of satisfactory treatment of the patient, reducing the time and number of interactions, such as physician visits, endured by the patient. This optimization includes selection of electrode polarities, electrode configurations stimulating parameter waveforms, temporal profile of stimulation magnitude, stimulation duty cycles, baseline stimulation magnitude, intermittent stimulation magnitude and timing, and other stimulation parameters.

Some embodiments relate to provision of signal processed sensory feedback signals to clinicians to augment their manual selection of optimum treatment magnitude and pattern. Sensory feedback signals provided to the clinician via a clinician-patient interface include but are not limited to tremor estimates, electromyography (EMG) signals, EEG signals, accelerometer signals, acoustic signals, peripheral nerve signals, cranial nerve signals, cerebral or cerebellar cortical signals, signals from basal ganglia, signals from other brain or spinal cord structures, and other signals.

In some embodiments, the invention provides modulation of treatment magnitude to compensate for predictable fluctuations in symptomatology and cognitive and neuromotor functionality. Such fluctuations include those due to, for example, the circadian cycle, postprandial and nutritional changes in symptomatology, and variations in plasma levels of pharmacologic agents.

In some embodiments, the invention provides prediction of future symptomatology, cognitive and neuromotor functionality, and treatment magnitude requirements. Such predictions can be based on preset, learned and real-time sensed parameters as well as input from the patient, physician or other person or system. In some embodiments, the input comprises electroencephalography (EEG), magnetoencephalography (MEG), optical imaging, Bold, magnetic resonance imaging (MRI), functional MRI (FMRI), transcranial doppler (TCD), CAT and other forms of neuronal inputs. In some embodiments, analyzing the input comprises digital signal processing, analog pattern recognition, etc. Some embodiments relate to a system for pattern recognition in a subject indicative of disease or neurological state comprising an output system, an input system and a control system, coupled between the input system and the output system. In some embodiments, the control system is a gated control system.

In some embodiments, the output delivery system comprises output comprising transcranial magnetic stimulation (TMS), transcranial direct current stimulation (tDCS), etc.

### BRIEF DESCRIPTION OF THE DRAWINGS

FIG. 1 illustrates, schematically, an illustrative system in accordance with an embodiment.

FIG. 2 illustrates, schematically, an output delivery system in accordance with an embodiment.

FIG. 3 illustrates, schematically, a method in accordance with an embodiment.

FIG. 4 illustrates a schematic block diagram of a generic computing device which may provide an operating embodiment in one or more embodiments.

### DETAILED DESCRIPTION OF THE INVENTION

A growing understanding of these techniques has led to the development of numerous diagnostic and therapeutic applications in neurology, cognitive neuroscience, clinical neurophysiology, psychiatric disorders, such as depression, hallucinations, obsessions, and drug craving; neurological diseases such as Parkinson's disease, or dystonia; neurorehabilitation, such as of aphasia or of hand function after stroke; and pain syndromes, such as caused by migraine, neuropathies, low back pain, or internal visceral diseases such as chronic pancreatitis or cancer.

For many conventional applications, effective brain stimulation techniques have included routine stimulation according to a prescribed schedule. For example, a particular therapeutic application may have the best results when repeated for several days (e.g., ten to twenty days) on a daily or even bi-daily regimen. Other applications may require more or less frequent stimulation and/or require different lengths of time over which the stimulation is repeated. For example, the prescribed length of time may vary from a couple of days, to several weeks, months or years. In some circumstances, the most effective treatment may involve an indefinite stimulation regimen. In particular, after the initial treatment (referred to as the induction phase), a subject may require (or respond more positively) by continuing with maintenance therapy for many months, and possibly indefinitely.

Embodiments of the invention relate to novel methods of correlating neurological states, including neurological disease or disorder states, with patterns produced by the brain activity of a subject experiencing the neurological state. Embodiments of the invention further relate to methods of analyzing the patterns produced by the brain activity of a subject experiencing a neurological disease or disorder and treating the subject according to the pattern by means that alter the subject's brain activity.

Embodiments of the invention also include systems to treat a subject suffering from a neurological disease or disorder based on the patterns produced by brain activity in the subject, wherein the system provides a means to alter the subject's brain activity as a function of the pattern. The system can include an output system, an input system and a control system that is coupled between the input system and the output system. The input system receives input indicative of brain activity in a subject suffering from a neurological disease or state and analyzes the input, for example, but not limited to, by way of an algorithm, to determine the presence or absence of a particular pattern correlated to a neurological disease or condition. The control system allows the analysis of information provided by the input system to be applied by the output system in the form of any output that alters the subject's brain activity. In some embodiments, the control system is a gated control system. In some embodiments, the output is provided by the output system as a multiplicity of doses. In further embodiments, the output is provided by the output system as a series of escalating doses.

In embodiments of the invention, the system is used to treat a subject suffering from a neurological disease or state is provided in a portable device.

In embodiments of the invention, the methods and systems disclosed herein can be used to treat a neurological disease or state. In other embodiments, the methods and systems disclosed herein can be used to prevent a neurological disease or state.

Embodiments of the invention also relate to methods of assessing and optimizing positions of a subject's brain for the administration of neuromodulatory stimuli. The methods can comprise identifying a first candidate position on the subject for administration of neuromodulatory stimulation, administering neuromodulatory stimulation to the subject, observing the subject for presentation of desired or undesired effects associated with delivery of the administered neurostimulation, and comparing the results to those results produced by delivery of neuromodulatory stimulation to a second candidate position. In some embodiments, the methods further comprise changing the position for administration of neuromodulatory stimulation based on comparison of the results. The changing of the position can be carried out by an operator. In some embodiments, the changing of the position can be controlled by the control system. In further embodiments, the changing of the position can be controlled by feedback loops in the controlled system. In some embodiments, the changing of the position can be carried out by a robotic device.

The embodiments of this invention could apply, but not limited, to mood disorders, anxiety disorders, physiological disorders, movement disorders, psychological diseases and disorders and neuroendocrine disorders.

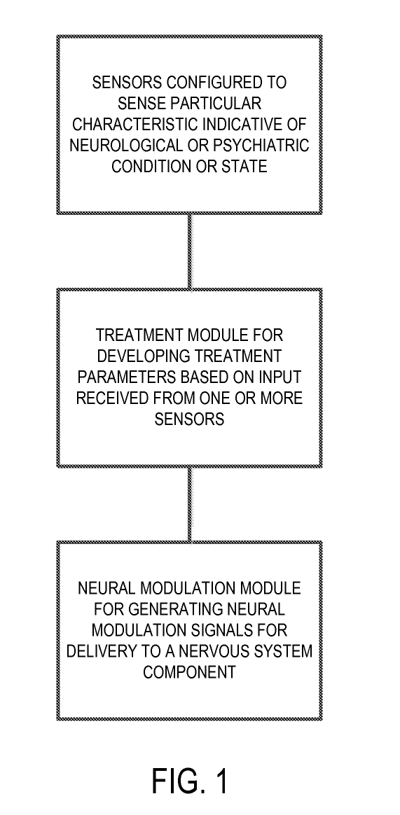

FIG. 1 shows schematically is an illustrative system in accordance with an embodiment. As shown, a plurality of sensors may be used to sense a particular characteristic indicative of a neurological or psychiatric condition or state. A treatment module receives input from the one or more sensors, and develops treatment parameters based on an input. A neural modulation module then generates a neural modulation signals for delivery to a nervous system component through one or more output devices in accordance with one or more developed treatment parameters.

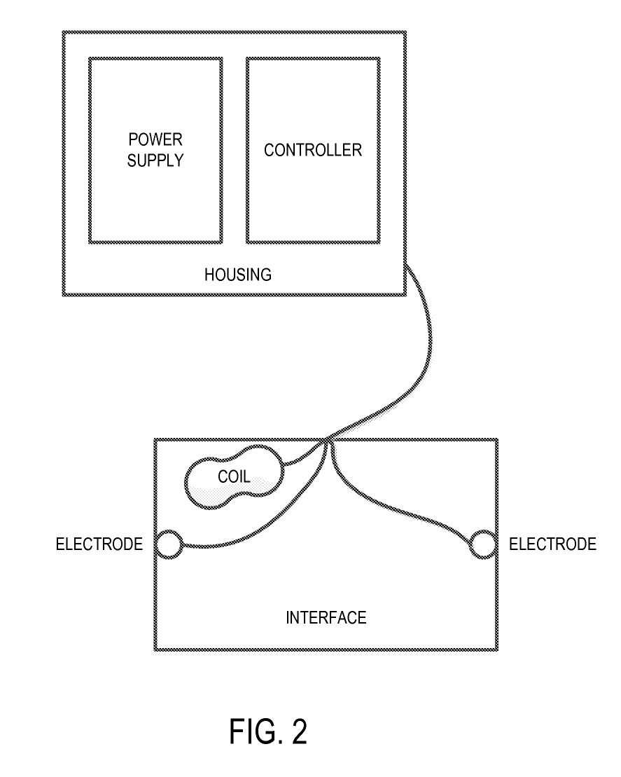

Referring to FIG. 2, shown schematically is an illustrative neural modulation module for delivering a neural modulation signal. In this illustrative example, an electromagnetic coil is connected to a power supply that energizes the coil. The power supply may comprise, for example, a housing containing a power source. The power source may be, for example, a battery, or alternatively a line filter, rectifier and power factor corrector to convert an AC line voltage to a suitable voltage for the system. A controller controls the operation of the electromagnetic coil to determine how the coil is energized (e.g. timing, duration, power, etc.). The controller may be embodied in a generic computing device having suitable interconnections to the neural modulation module.

Electrodes provided on an interface may be placed on a subject to deliver the neural modulation signal. U.S. application Ser. No. 12/312,931, filed Dec. 1, 2007 (published as US 2010/0210894 A1) and U.S. Provisional Application No. 60/872,207, filed Dec. 1, 2006, both entitled TRANSCRANIAL MAGNETIC STIMULATION (TMS) METHODS AND APPARATUS, disclose further details regarding an illustrative system for delivering TMS. Both applications are incorporated herein by reference in their entirety. Figures from US 2010/0210894 A1 show an illustrative system for delivering TMS and are attached hereto as Appendix A.

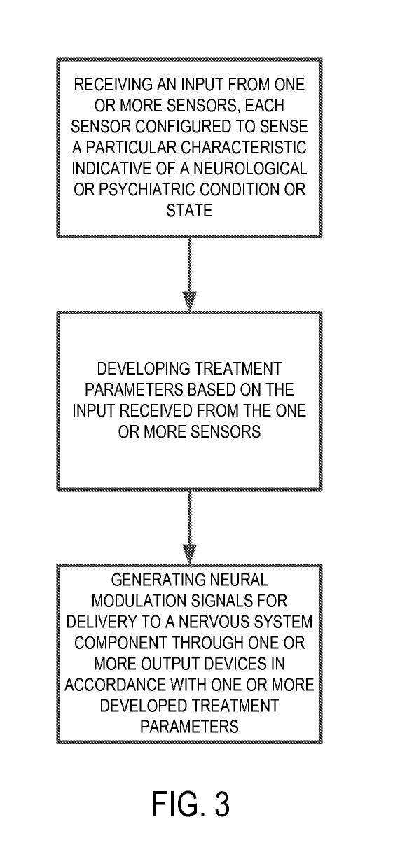

Now referring to FIG. 3, shown is an illustrative method in accordance with an embodiment. As shown, a method of modulating activity of a nervous system component, comprises first receiving an input from one or more sensors, each sensor configured to sense a particular characteristic indicative of a neurological or psychiatric condition or state. The method then develops treatment parameters based on the input received from the one or more sensors. Finally, the method generates neural modulation signals for delivery to a nervous system component through one or more output devices in accordance with one or more developed treatment parameters.

Further details of methods and systems in accordance with various embodiments of the invention are now described below.

### Neurological State and Activation Patterns

The brain is viewed as a series of electrical circuits that provide vital functions in living organisms. Accordingly, brain activity can be monitored by the electrical activity that occurs during normal functions that allow organisms to survive and live. Similarly, brain activity can be altered by malfunctions that are indicative of neurological, psychological, or physiological disorders and states that affect the brain. As a consequence, distinct brain activation patterns can be produced. For example, it has been demonstrated that dyslexic children experience increased activation patterns in the frontal and temporal lobes relative to children with normal reading ability (Arms, et al. 2007. Journal of Integrative Neuroscience 6:175-190, which is incorporated herein by reference in its entirety). Similarly, other neurological disorders can be distinguished from normal brain activity by analysis and recognition of distinct patterns. Analysis and recognition of distinct brain activity patterns can be done with the aid of a computer, or by direct inspection (as when EEG is interpreted).

Accordingly, embodiments of the invention relate to methods of treating a subject suffering from a neurological disease or state, comprising analyzing the subject's brain activity for patterns associated with the neurological disease or state and treating the subject according to the pattern. The neurological disease or state can be at least one of: headache, migraine headache, epilepsy, Parkinson's disease, Huntington's disease, Parkinsonism, rigidity, hemiballism, choreoathetosis, dystonia, akinesia, bradykinesia, hyperkinesia, other movement disorders, ticks, seizure disorders, brain injuries, and the like.

In other embodiments, methods of treating a subject suffering from a psychiatric disease or disorder are provided comprising analyzing the subject's brain activity for pattern recognition associated with the psychiatric disease or disorder and treating the subject according to the recognized pattern. The psychiatric disease or disorder can be at least one of: depression, bipolar disorder, other affective disorder, anxiety, phobia, schizophrenia, multiple personality disorder, obsessive compulsive disorder (OCD) and its spectrum, post traumatic stress disorder (PTSD), social anxiety disorder, panic disorder, chronic pain syndrome, insomnia, chronic fatigue syndrome, stress, substance abuse, attention deficit hyperactivity disorder, impaired control of aggression, impaired control of sexual behavior, and the like.

### Inputs and Input Systems

Analysis of the brain activity in a subject suffering from a neurological or psychological disease or state can include analysis of signals indicative of the subject's brain activity. The input can be detected by sources that provide temporal and/or spatial resolution of ongoing brain activity. For example, signal input indicative of the subject's brain activity can be obtained from any device capable of detecting electrical activity in the brain. In other embodiments, the input can be obtained by any device capable of detecting changes in metabolic rate or blood flow. Exemplary devices include, but are not limited to, commercially available systems, such as, electroencephalography (EEG), magnetoencephalography (MEG), optical imaging, near-infrared spectroscopy (NIRS), magnetic resonance imaging (MRI), functional MRI (fMRI), transcranial doppler (TCD), and the like.

The signal input can be detected by any non-invasive means. For example, in non-invasive embodiments, signal input can be obtained by placing electrodes about a subject's head to detect electric signals indicative of brain activity. In other embodiments, the signal input can be obtained by surgically implanting electrodes on the surface or within the depth of the subject's brain. In some embodiments, the signal input is detected by electrodes placed subdurally.

The signal input system can comprise any number of commercially available systems. For example, in some embodiments, a subject has a number of electrodes (1, 2, 3, . . . n) attached to his or her scalp in a conventional manner. The electrodes are used to sense brain activity to provide a signal input. The electrical brain activity sensed by electrodes is converted into an electrical signal that is transmitted to an input control unit. The electrodes can be located on the patient's scalp in accordance, for example, with the international 10-20 system.

In some embodiments of the invention, the transmitted electrical signal is transmitted to a preamplifier upstream of the input control unit. In some embodiments, the preamplifier has at least 12 channels, and at least 12 electrodes are used. For example, the preamplifier can have 12, 13, 14, 15, 16, 17, 18 or more channels, and 12, 13, 14, 15, 16, 17, 18 or more electrodes are used. In other embodiments, the preamplifier has at least 18 channels, and at least 18 electrodes are used. However, one skilled in the art will appreciate that the amplifier can be provided with additional channels and additional electrodes can be provided. The preamplifier can be constructed and arranged to be small and lightweight. In some embodiments, the preamplifier can be located such that it lies at the nape of the subject's neck. The preamplifier is constructed such that it is small and provides a relatively high gain close to the electrodes with a limited bandpass and relatively high common mode rejection ratio. In some embodiments, the preamplifier can use surface mount components. The output of preamplifier can be carried by a conductor to the input control unit.

The input control unit has appropriate processing circuitry for processing the signal to provide a visual indication or record of brain wave activity. In some embodiments, a strip chart recorder is provided to provide a paper record of the signal input.

### Pattern Recognition Methods

In embodiments of the invention, methods and systems are provided for analyzing a subject's brain activity and performing pattern recognition of the brain activity based upon a disease state or disorder. Any region of the brain, including the cortex, temporal lobe, frontal lobe, tempoparietal lobe, and the like can be analyzed for brain activity. The disease state can be at least one of, but is not limited to, headache, migraine headache, spreading depression (SD) of migraine-related cortical activity, epilepsy, Parkinson's disease, Huntington's disease, Parkinsonism, rigidity, hemiballism, choreoathetosis, dystonia, akinesia, bradykinesia, hyperkinesia, other movement disorders, ticks, seizure disorders, brain injuries, and the like. The disorder can be at least one of, but is not limited to, depression, bipolar disorder, other affective disorder, anxiety, phobia, schizophrenia, multiple personality disorder, obsessive compulsive disorder (OCD) and its spectrum, post traumatic stress disorder (PTSD), social anxiety disorder, panic disorder, chronic pain syndrome, insomnia, tinnitus, learning disorder, chronic fatigue syndrome, stress, substance abuse, attention deficit hyperactivity disorder, impaired control of aggression, impaired control of sexual behavior, and the like.

In some embodiments, a subject suffering from epilepsy is diagnosed by analysis of the subject's brain activity for epileptiform discharges. The epileptiform discharges can be analyzed to identify seizure type.

In some embodiments, a subject suffering from depression is diagnosed by detection and analysis of brain activity variance in frontal cortical regions.

In some embodiments, a subject suffering from auditory hallucination and/or schizophrenia is diagnosed by detection and analysis of brain activity variance in the tempoparietal cortex.

In some embodiments, a subject suffering from obsessive compulsive disorder (OCD) or Tourette's syndrome is diagnosed by detection and analysis of brain activity variance in the cortical and supplementary motor area.

In some embodiments, a subject suffering from tinnitus is diagnosed by detection and analysis of excessive brain activity in the auditory cortex.

In some embodiments of the invention, methods are provided to detect patterns in the underlying brain activity of a subject suffering from a disease or disorder, comprising receiving a signal input indicative of underlying brain activity in the subject and analyzing identifiable features in the underlying brain activity. Identifiable features in the underlying brain activity include, but are not limited to, detectable phases, maximal or minimal voltages, changes in frequency or amplitude of signal, changes in location of signal source, and the like.

### Control Systems

In embodiments of the invention, a control system is provided between the input system and output system. The control system receives the input signals provided by the input system and uses those signals, either automatically or under control of an operator, to control the output system.

In some embodiments, the control system is a gated control system. For example, the control system can control a “gate” that opens and closes to allow output to be delivered to the subject based on set thresholds of signal input. Output parameters such as, for example, amplitude, frequency, or signal, can be controlled in this manner. In some embodiments, the control system can also control how quickly the gate closes once the input level has dropped below the threshold, or inversely, how quickly the gate opens once the input level. In some embodiments, the control system contains a hold control which allows a minimum amount of time the gate will stay open even if signal input temporarily falls below set thresholds.

In some embodiments, the signal output can be reduced in amplitude and/or intensity (“attenuation”) rather than completely stopped when the gate is closed. The amount of attenuation when the gate is closed can be set by a range control. For example, in embodiments where attenuation is complete, no signal will pass when the gate is closed. In some embodiments, complete attenuation is not desired and the range can be changed such that low amplitude and/or intensity levels of signal output are delivered to the subject when the gate is closed.

In some embodiments, the gate is not controlled by signal input but is instead controlled by a preprogrammed pattern resulting in a precisely controlled opening and closing of the gate.

In some embodiments, gated control systems implement hysteresis, that is, they have two thresholds. A first upper threshold allows the gate to be opened, and a second lower threshold allows the gate to be closed. As a result, once signal input drops below the lower threshold, it must rise to the upper threshold for the gate to open.

In some embodiments, the control system can time the neurostimulatory output based upon patterns of signal input indicative of phases of brain activity in the subject. For example, in some embodiments, the control system can adjust or control delivery of an individual neuromodulation stimulus or series of neuromodulation stimuli based upon coordination with phases of neuronal discharge. In other embodiments, the control system can adjust or control delivery of an individual neuromodulation stimulus or series of neuromodulation stimuli based upon coordination with patterns of discharge.

In some embodiments, the control system includes a computer having a keyboard, a display, and a mass storage device connected thereto. The display can be, for example, a cathode ray tube or flat panel LCD display. The mass storage device can be, but is not limited to, any kind of magnetic or optical medium mass storage device such as a disk drive or a tape drive. The mass storage device can be used for storing programs to be executed by the computer as well as for storing input information provided by the input system and output information provided by the output system.

In some embodiments, the control system provides a safety shutdown control. For example, in some embodiments where neurostimulatory procedures are administered as repetitive output pulses, the output pulses can cause seizure activity in the brain of the subject undergoing treatment. In some cases, the patient can actually go into a full body seizure. To avoid seizure, the control system is used to monitor the input signals and to turn off the output system by providing an off control signal if seizure activity is detected. Seizure activity can be detected in several ways. In some embodiments, an operator can detect changes in the input signal pattern exhibiting recruitment or the onset of a seizure discharge and can activate a “stop” button, for example, in the control system to shut off the output system before the seizure actually occurs. In other embodiments, the input signal can be analyzed by pattern recognition programs running on the computer. The operator can detect abnormal activity via computer analysis, and the control system can be used to shut down the output system before a seizure results. In some other embodiments, conventionally available computer software that automatically detects the onset of seizures using specified criteria can be used automatically in a control program running on the computer or in conjunction with the operator to shut off the output system. Exemplary seizure detection software is available from, for example, Stellate, Inc., and the like.

In embodiments of the invention, the control system can be used to recognize when the brain has been placed in a desired state as a result of the neurostimulatory procedure or can be used to recognize when the brain has been removed from an undesired state by the neurostimulatory procedure. The subject's input signals can be monitored while the treatment is being applied, and when the patient's input signals no longer contains abnormal characteristics or conform to patterns associated with neurological and psychological diseases and disorders, the procedure can be stopped. In some embodiments, an operator can be used to determine if and when a treatment procedure has been successful.

In some embodiments, the invention can be used to determine when a patient's brain activity has reached a desired state. For example, if a normal input signal indicative of normal brain activity is the target for a particular subject, then embodiments of the invention can be used to monitor the patient's input signal as the treatment is applied, and this can be used to determine when the patient's brain activity has reached the normal or desired state. In some embodiments, the operator can stop the procedure when the patient's brain activity is in the desired state.

### Output Systems

The output system can be any system that delivers a neurostimulatory output to a subject in need of neuromodulation or to a subject suffering from a neurological or psychiatric disease or disorder. In some embodiments, the output system comprises a transcranial magnetic stimulation (TMS) system. In other embodiments, the output system comprises a transcranial direct current stimulation (tDCS) system. Additional exemplary systems include, but are not limited to, visual stimulation (e.g. flash of light), auditory stimulation (e.g. loud click), electrical stimulation of one or more peripheral or cranial nerves, near-infrared transcranial stimulation.

### Transcranial Magnetic Stimulation (TMS) Systems

Embodiments of a TMS system comprise a device that includes one or more electromagnetic coils that, when energized, emit a magnetic field either as a single pulse of magnetic energy or as magnetic energy that fluctuates in intensity or polarity. The magnetic field can be applied to the brain of a subject to induce an electric current therein, which, in turn, can be used to treat a variety of neurological and psychiatric diseases and states as disclosed herein. The electromagnetic coil can be housed in an interface that facilitates quick and easy positioning of the coil proximate to the subject's brain. A controller can be provided that runs the electromagnetic coil according to a defined, operating protocol that further simplifies use of the device.

In embodiments of the invention, the parameters of the TMS procedure can be adjusted depending upon the subject's brain activity patterns as indicated by the input signals. For example, parameters including, but not limited to, the strength of the magnetic field being applied, the frequency of the magnetic pulses, and the location of the one or more TMS coils on the subject's head can be adjusted in response to the changes induced in the subject's brain activity as a result of a TMS procedure. The placement of the one or more TMS coils can be adjusted, for example, by an operator holding the one or more coils. In some embodiments, a robotic arm controlled by the operator can be used to hold the one or more TMS coils on the subject's head.

Transcranial Direct Current Stimulation (tDCS) Systems

Embodiments of a tDCS system comprise one, two, three, four or more pairs of electrodes, wherein each pair includes a positively-charged electrode (“anode”) and a negatively-charged electrode (“cathode”). The pairs of electrodes can be applied bilaterally to a subject in accordance with the cortex areas corresponding to the disease or disorder to be treated. Positively charged tDCS (“anodal tDCS”) or negatively charged tDCS (“cathodal tDCS”) can be applied intermittently over a period of at least 15, 20, 25, 30, 45 or 60 minutes by a constant current stimulator.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Pattern Recognition of Seizure

A group of subjects clinically diagnosed with epilepsy are individually evaluated using an EEG system for monitoring brain activity in the frontal cortical region. A control group of healthy subjects are individually evaluated in parallel using an EEG system. The EEG input is collected and analyzed, and a common pattern or patterns of epileptiform discharges is observed for the group of epileptic subjects.

### Example 2

### Treatment of Seizure

A subject is evaluated using an embodiment of the invention in which EEG is detected as signal input and TMS is administered as either single pulsed or repetitive neuromodulatory stimulation output. The subject's brain activity is monitored using EEG, and the EEG input is analyzed. When the subject experiences seizure, a pattern of brain activity associated with seizure is identified, and TMS is administered to the subject by an operator or software following a seizure treatment protocol. The TMS is administered over a wide range of frequencies (0.5 Hz to over 100 Hz), periods encompassing single sessions to months of daily sessions, pulse widths and polarities. Over the course of treatment, the patient's seizure symptoms diminish, as evaluated by clinical factors as well as EEG analysis of brain activity.

### Example 3

### Optimization of Neurostimulatory Location

A group of subjects clinically diagnosed with epilepsy are individually evaluated using an EEG system for monitoring brain activity. A second group of subjects clinically diagnosed with epilepsy are individually evaluated in parallel using an EEG system. The EEG input is collected and analyzed, and a common pattern or patterns of epileptiform discharges is observed for both groups of epileptic subjects when the subjects suffer epileptic seizures. The first group is treated with TMS according to a seizure treatment protocol, and the TMS is administered over a wide range of frequencies, periods, pulse widths, and polarities to a first cortical location on each subject within the group. The second group is treated with TMS according to a seizure treatment protocol, and the TMS is administered over a wide range of frequencies, periods, pulse widths, and polarities to a second cortical location on each subject within the group. Over the course of treatment, it is observed that subjects in the second group exhibit recover from seizure in significantly faster time relative to those of subjects in the first group.

### Example 4

### Assessing Therapeutic Response

A subject is evaluated using an embodiment of the invention in which EEG is detected as signal input and TMS is administered as neuromodulatory stimulation output. The subject's brain activity is monitored using EEG, and the EEG input is analyzed. When the subject experiences seizure, a pattern of brain activity associated with seizure is identified, and TMS is administered to the subject by an operator or software following a seizure treatment protocol. The TMS is administered over a wide range of frequencies, periods, pulse widths, and polarities. Over the course of treatment, the patient's seizure symptoms diminish, as evaluated by clinical factors as well as EEG analysis of brain activity. Once the patient's EEG results indicate abatement of the seizure, the operator or software ceases to administer TMS.

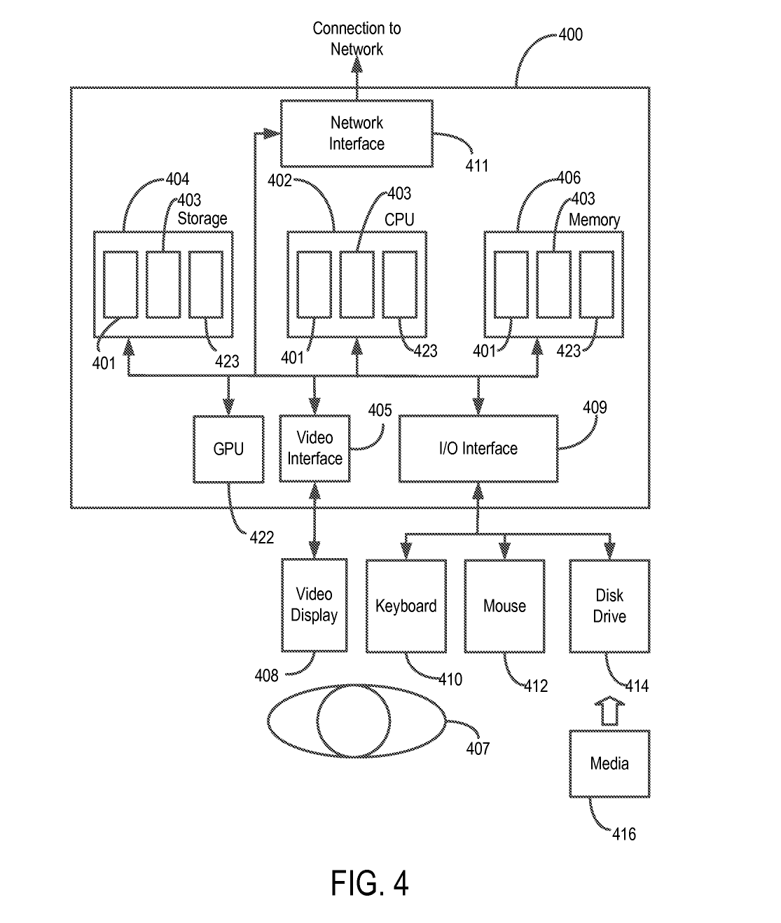

FIG. 4 shows a schematic block diagram of a generic computing device which may provide an operating embodiment in one or more embodiments. A suitably configured computer device, and associated communications networks, devices, software and firmware may provide a platform for enabling one or more embodiments as described above. By way of example, FIG. 4 shows a generic computer device 400 that may include a central processing unit (“CPU”) 402 connected to a storage unit 404 and to a random access memory 406. The CPU 402 may process an operating system 401, application program 403, and data 423. The operating system 401, application program 403, and data 423 may be stored in storage unit 404 and loaded into memory 406, as may be required. Computer device 400 may further include a graphics processing unit (GPU) 422 which is operatively connected to CPU 402 and to memory 406 to offload intensive image processing calculations from CPU 402 and run these calculations in parallel with CPU 402. An operator 407 may interact with the computer device 400 using a video display 408 connected by a video interface 405, and various input/output devices such as a keyboard 410, pointer 412, and storage 414 connected by an I/O interface 409. In known manner, the pointer 412 may be configured to control movement of a cursor or pointer icon in the video display 408, and to operate various graphical user interface (GUI) controls appearing in the video display 408. The computer device 400 may form part of a network via a network interface 411, allowing the computer device 400 to communicate with other suitably configured data processing systems or circuits, such as the extrusion logic motor circuit of the apparatus described above. One or more different types of sensors 430 connected via a sensor interface 432 may be used to search for and sense input from various sources. The sensors 430 may be built directly into the generic computer device 400, or optionally configured as an attachment or accessory to the generic computer device 400.

Thus, in an aspect, there is provided a method of modulating activity of a nervous system component, comprising: receiving an input from one or more sensors, each sensor configured to sense a particular characteristic indicative of a neurological or psychiatric condition or state; developing treatment parameters based on the input received from the one or more sensors; and generating neural modulation signals for delivery to a nervous system component through one or more output devices in accordance with one or more developed treatment parameters.

In an embodiment, the one or more sensors are adapted to sense brain activity, and the method further comprises analyzing a subject's brain activity for patterns associated with the neurological or psychiatric condition or state.

In another embodiment, the one or more sensors comprise electroencephalography (EEG), magnetoencephalography (MEG), optical imaging, near-infrared spectroscopy (NIRS), magnetic resonance imaging (MRI), functional MRI (FMRI), and transcranial doppler (TCD).

In another embodiment, analyzing the subject's brain comprises performing pattern recognition of the brain activity based upon a disease state or disorder.

In another embodiment, analyzing the subject's brain comprises analyzing one or more regions of the subject's brain including the cortex, temporal lobe, frontal lobe and the temporarietal lobe for regional brain activity.

In another embodiment, the disease state or disorder includes one or more of headache, migraine headache, epilepsy, Parkinson's disease, Huntington's disease, Parkinsonism, rigidity, hemiballism, choreoathetosis, dystonia, akinesia, bradykinesia, hyperkinesia, other movement disorders, ticks, seizure disorders, brain injuries, depression, bipolar disorder, other affective disorder, anxiety, phobia, schizophrenia, multiple personality disorder, obsessive compulsive disorder (OCD), post traumatic stress disorder (PTSD), social anxiety disorder, panic disorder, chronic pain syndrome, insomnia, tinnitus, learning disorder, chronic fatigue syndrome, stress, substance abuse, attention deficit hyperactivity disorder, impaired control of aggression, or impaired control of sexual behavior.

In another embodiment, developing treatment parameters based on the input received from the one or more sensors comprises utilizing a control system to control one or more output parameters in dependence upon the recognized disease state or disorder.

In another embodiment, the method further comprises providing a gated control system to control an output signal for delivery to the subject in dependence upon the input received from the one or more sensors.

In another embodiment, the gated control system is adapted to control various output parameters including amplitude, frequency, signal type, and speed of gate opening or closure.

In another embodiment, the method further comprises providing a safety shutdown control for automatically shutting down the output signal upon reaching an input signal level threshold.

In another aspect, there is provide a system for modulating activity of a nervous system component, comprising: one or more sensors, each sensor configured to sense a particular characteristic indicative of a neurological or psychiatric condition or state; a treatment module for developing treatment parameters based on an input received from the one or more sensors; and a neural modulation module for generating neural modulation signals for delivery to a nervous system component through one or more output devices in accordance with one or more developed treatment parameters.

In an embodiment, the one or more sensors are adapted to sense brain activity, and the system is adapted to analyze a subject's brain activity for patterns associated with the neurological or psychiatric condition or state.

In another embodiment, the one or more sensors comprise electroencephalography (EEG), magnetoencephalography (MEG), optical imaging, near-infrared spectroscopy (NIRS), magnetic resonance imaging (MRI), functional MRI (FMRI), and transcranial doppler (TCD).

In another embodiment, the system is further adapted to analyze the subject's brain by performing pattern recognition of the brain activity based upon a disease state or disorder.

In another embodiment, the system is further adapted to analyze one or more regions of the subject's brain including the cortex, temporal lobe, frontal lobe and the temporarietal lobe for regional brain activity.

In another embodiment, the disease state or disorder includes one or more of headache, migraine headache, epilepsy, Parkinson's disease, Huntington's disease, Parkinsonism, rigidity, hemiballism, choreoathetosis, dystonia, akinesia, bradykinesia, hyperkinesia, other movement disorders, ticks, seizure disorders, brain injuries, depression, bipolar disorder, other affective disorder, anxiety, phobia, schizophrenia, multiple personality disorder, obsessive compulsive disorder (OCD), post traumatic stress disorder (PTSD), social anxiety disorder, panic disorder, chronic pain syndrome, insomnia, tinnitus, learning disorder, chronic fatigue syndrome, stress, substance abuse, attention deficit hyperactivity disorder, impaired control of aggression, or impaired control of sexual behavior.

In another embodiment, the treatment module is adapted to develop treatment parameters based on the input received from the one or more sensors comprises utilizing a control system to control one or more output parameters in dependence upon the recognized disease state or disorder.

In another embodiment, the system is further adapted to provide a gated control system to control an output signal for delivery to the subject in dependence upon the input received from the one or more sensors.

In another embodiment, the system is further adapted to control various output parameters of the gated control system, including amplitude, frequency, signal type, and speed of gate opening or closure.

In another embodiment, the system is further adapted to provide a safety shutdown control for automatically shutting down the output signal upon reaching an input signal level threshold.

While illustrative embodiments of the invention have been described above, it will be appreciate that various changes and modifications may be made without departing from the scope of the present invention.

1. A method of modulating activity of a nervous system component, comprising:
receiving an input from one or more sensors, each sensor configured to sense a particular characteristic indicative of a neurological or psychiatric condition or state;
developing treatment parameters based on the input received from the one or more sensors; and
generating neural modulation signals for delivery to a nervous system component through one or more output devices in accordance with one or more developed treatment parameters.

2. The method of claim 1, wherein the one or more sensors are adapted to sense brain activity, and the method further comprises analyzing a subject's brain activity for patterns associated with the neurological or psychiatric condition or state.

3. The method of claim 2, wherein the one or more sensors comprise electroencephalography (EEG), magnetoencephalography (MEG), optical imaging, near-infrared spectroscopy (NIRS), magnetic resonance imaging (MRI), functional MRI (FMRI), and transcranial doppler (TCD).

4. The method of claim 2, wherein analyzing the subject's brain comprises performing pattern recognition of the brain activity based upon a disease state or disorder.

5. The method of claim 4, wherein analyzing the subject's brain comprises analyzing one or more regions of the subject's brain including the cortex, temporal lobe, frontal lobe and the temporarietal lobe for regional brain activity.

6. The method of claim 4, wherein the disease state or disorder includes one or more of headache, migraine headache, epilepsy, Parkinson's disease, Huntington's disease, Parkinsonism, rigidity, hemiballism, choreoathetosis, dystonia, akinesia, bradykinesia, hyperkinesia, other movement disorders, ticks, seizure disorders, brain injuries, depression, bipolar disorder, other affective disorder, anxiety, phobia, schizophrenia, multiple personality disorder, obsessive compulsive disorder (OCD), post traumatic stress disorder (PTSD), social anxiety disorder, panic disorder, chronic pain syndrome, insomnia, tinnitus, learning disorder, chronic fatigue syndrome, stress, substance abuse, attention deficit hyperactivity disorder, impaired control of aggression, or impaired control of sexual behavior.

7. The method of claim 4, wherein developing treatment parameters based on the input received from the one or more sensors comprises utilizing a control system to control one or more output parameters in dependence upon the recognized disease state or disorder.

8. The method of claim 7, wherein the method further comprises providing a gated control system to control an output signal for delivery to the subject in dependence upon the input received from the one or more sensors.

9. The method of claim 8, wherein the gated control system is adapted to control various output parameters including amplitude, frequency, signal type, and speed of gate opening or closure.

10. The method of claim 9, further comprising providing a safety shutdown control for automatically shutting down the output signal upon reaching an input signal level threshold.

11. A system for modulating activity of a nervous system component, comprising:
one or more sensors, each sensor configured to sense a particular characteristic indicative of a neurological or psychiatric condition or state;
a treatment module for developing treatment parameters based on an input received from the one or more sensors; and
a neural modulation module for generating neural modulation signals for delivery to a nervous system component through one or more output devices in accordance with one or more developed treatment parameters.

12. The system of claim 11, wherein the one or more sensors are adapted to sense brain activity, and the system is adapted to analyze a subject's brain activity for patterns associated with the neurological or psychiatric condition or state.

13. The system of claim 12, wherein the one or more sensors comprise electroencephalography (EEG), magnetoencephalography (MEG), optical imaging, near-infrared spectroscopy (NIRS), magnetic resonance imaging (MRI), functional MRI (FMRI), and transcranial doppler (TCD).

14. The system of claim 12, wherein the system is further adapted to analyze the subject's brain by performing pattern recognition of the brain activity based upon a disease state or disorder.

15. The system of claim 14, wherein the system is further adapted to analyze one or more regions of the subject's brain including the cortex, temporal lobe, frontal lobe and the temporarietal lobe for regional brain activity.

16. The system of claim 14, wherein the disease state or disorder includes one or more of headache, migraine headache, epilepsy, Parkinson's disease, Huntington's disease, Parkinsonism, rigidity, hemiballism, choreoathetosis, dystonia, akinesia, bradykinesia, hyperkinesia, other movement disorders, ticks, seizure disorders, brain injuries, depression, bipolar disorder, other affective disorder, anxiety, phobia, schizophrenia, multiple personality disorder, obsessive compulsive disorder (OCD), post traumatic stress disorder (PTSD), social anxiety disorder, panic disorder, chronic pain syndrome, insomnia, tinnitus, learning disorder, chronic fatigue syndrome, stress, substance abuse, attention deficit hyperactivity disorder, impaired control of aggression, or impaired control of sexual behavior.

17. The system of claim 14, wherein the treatment module is adapted to develop treatment parameters based on the input received from the one or more sensors comprises utilizing a control system to control one or more output parameters in dependence upon the recognized disease state or disorder.

18. The system of claim 17, wherein the system is further adapted to provide a gated control system to control an output signal for delivery to the subject in dependence upon the input received from the one or more sensors.

19. The system of claim 18, wherein the system is further adapted to control various output parameters of the gated control system, including amplitude, frequency, signal type, and speed of gate opening or closure.

20. The system of claim 19, wherein the system is further adapted to provide a safety shutdown control for automatically shutting down the output signal upon reaching an input signal level threshold.
